(19) United States
(12) Patent Application Publication
(10) Pub. No.: US 2023/0059521 A1
(43) Pub. Date: Feb. 23, 2023

(54) METHODS OF TREATING OR PREVENTING A VIRAL INFECTION USING BACTERIOPHAGES

(71) Applicant: Atossa Therapeutics, Inc., Seattle, WA (US)

(72) Inventors: Shu-Chih CHEN, Seattle, WA (US); Steven Carl QUAY, Seattle, WA (US)

(21) Appl. No.: 17/795,386

(22) PCT Filed: Mar. 23, 2021
(86) PCT No.: PCT/US21/23615

§ 371 Date: Jul. 26, 2022

Related U.S. Application Data
(60) Provisional application No. 63/000,956, filed on Mar. 27, 2020.

(51) Int. Cl. A61K 35/76; A61P 31/14; C12N 7/00; A01N 63/40; A01P 1/00; A61P 31/18

(52) U.S. Cl. CPC A61K 35/76; A61P 31/14; C12N 7/00; A01N 63/40; A01P 1/00; A61P 31/18; C12N 2770/18071; C12N 2795/14133; C12N 2795/14121; C12N 2795/14171; C12N 2760/20171; C12N 2740/16071; C12N 2770/24271; C12N 2760/14171

(57) ABSTRACT

Described herein are compositions for treating or preventing a viral infection comprising bacteriophages that bind to the virus and block or inhibit viral entry into a host cell. Bacteriophage libraries may be screened to identify bacteriophages that bind to a virus of interest, and the identified bacteriophages may be used to treat or prevent an infection caused by the virus of interest. Also described herein are methods of treating or preventing a viral infection by administering a bacteriophage composition to a subject in vivo or to a surface in vitro. The bacteriophages in the composition may bind to the virus and inhibit viral entry into a host cell, thereby reducing the infectivity of the virus. Reducing the infectivity of the virus may treat or prevent the viral infection.

### CROSS-REFERENCE

The present application claims the benefit of U.S. Provisional Application No. 63/000,956, entitled “USES OF PROKARYOTIC VIRUSES IN THE THERAPEUTIC AND PREVENTIVE OF DISEASES CAUSED BY EUKARYOTIC VIRUSES,” filed on Mar. 27, 2020, which application is herein incorporated by reference in its entirety for all purposes.

### BACKGROUND

In recent years the serious threat posed by virus to worldwide public health has been highlighted. RNA viral diseases are responsible for the vast majority of viral morbidity and mortality of viral diseases of mankind, including AIDS, hepatitis, coronavirus or rhinovirus infections of the respiratory tract, flu, measles, polio and others. Coronaviruses are a large family of viruses that cause illness ranging from the common cold to more severe diseases such as Middle East Respiratory Syndrome (MERS-CoV) and Severe Acute Respiratory Syndrome (SARS-CoV). A novel coronavirus (nCoV), SARS-CoV-2, is a new strain that has not been previously identified in humans. SARS-CoV-2 is actually closer to the bat virus, sharing 96% of its genome sequence, compared to about 86% with SARS-CoV. Outbreaks and clusters of the disease (Covid-19) have since been observed in Asia, Europe, Australia, Africa and the Americas.

Currently there is no effective therapy or prevention for many viral diseases. Thus, there exists a need for an effective prevention or amelioration of virus-mediated diseases.

### SUMMARY

In various aspects, the present disclosure provides a method of reducing the infectivity of a virus in a subject, the method comprising: administering a composition comprising a bacteriophage to the subject, binding the bacteriophage to the virus, inhibiting invasion of the virus into a cell of the subject, and reducing the infectivity of the virus.

In some aspects, the virus is selected from the group consisting of a coronavirus, a human immunodeficiency virus, a herpes simplex virus, a hepatitis virus, a Marburg virus, an Ebola virus, a rhinovirus, an influenza virus, an avian influenza virus, a rotavirus, a norovirus, a dengue virus, a rabies virus, a mononucleosis virus, a human papillomavirus, a rubeola virus, a rubella virus, a zika virus, a varicella virus, and a poliovirus. In some aspects, the coronavirus is SARS-CoV-2, SARS-CoV, MERS-CoV, OC43, or HKU1.

In some aspects, the composition is administered by inhalation, parenteral administration, intravenous administration, intranasal administration, oral administration, or topical administration. In some aspects, the composition is administered by inhalation via nebulization. In some aspects, the method comprises delivering at least 10⁵, at least 10⁶, at least 10⁷, at least 10⁸, at least 10⁹, at least 10¹⁰, at least 10¹¹, or at least 10¹² bacteriophage particles to the subject. In some aspects, the method comprises delivering at least 10⁹ bacteriophage particles to the subject. In some aspects, the subject is a human, a non-human animal, a plant, or a single-celled eukaryote.

In some aspects, the composition comprises at least 2, at least 10, at least 20, at least 50, or at least 100 bacteriophage variants capable of binding to the virus. In some aspects, the composition comprises at least 50 bacteriophage variants capable of binding to the virus. In some aspects, the bacteriophage binds to the virus with an average dissociation constant of no more than 100 nM.

In some aspects, reducing the infectivity of the virus comprises inhibiting interactions between the virus and the cell of the subject. In some aspects, reducing the infectivity of the virus comprises binding the bacteriophage to the virus. In some aspects, the bacteriophage binds to a coat protein of the virus. In some aspects, the method further comprises treating an infection caused by the virus. In some aspects, the method further comprises preventing an infection caused by the virus.

In various aspects, the present disclosure provides a method of reducing the infectivity of a virus on a surface, the method comprising: applying a composition comprising a bacteriophage to the surface, binding the bacteriophage to the virus, inhibiting invasion of the virus into a host cell, and reducing the infectivity of the virus.

In some aspects, the host cell is a eukaryotic host cell. In some aspects, the virus is selected from the group consisting of a coronavirus, a human immunodeficiency virus, a herpes simplex virus, a hepatitis virus, a Marburg virus, an Ebola virus, a rhinovirus, an influenza virus, an avian influenza virus, a rotavirus, a norovirus, a dengue virus, a rabies virus, a mononucleosis virus, a human papillomavirus, a rubeola virus, a rubella virus, a zika virus, a varicella virus, and a poliovirus. In some aspects, the coronavirus is SARS-CoV-2, SARS-CoV, MERS-CoV, OC43, or HKU1. In some aspects, the composition at least 2, at least 10, at least 20, at least 50, or at least 100 bacteriophage variants capable of binding to the virus. In some aspects, the composition comprises at least 50 bacteriophage variants capable of binding to the virus.

In some aspects, the surface is a handle, a button, a switch, a seat, a counter, a floor, a wearable item, a body part, or an object. In some aspects, the composition is applied to the surface at a concentration of from 10⁵ to about 10¹² bacteriophage particles per mL. In some aspects, the composition is applied to the surface at a concentration of from 10⁶ to about 10¹¹ bacteriophage particles per mL. In some aspects, the composition is applied to the surface at a concentration of from 10⁹ to about 10¹⁰ bacteriophage particles per mL. In some aspects, the bacteriophage binds to the virus with an average dissociation constant of no more than 100 nM.

In some aspects, reducing the infectivity of the virus comprises inhibiting interactions between the virus and the host cell. In some aspects, reducing the infectivity of the virus comprises binding the bacteriophage to the virus. In some aspects, the bacteriophage binds to a coat protein of the virus. In some aspects, the method further comprises preventing an infection caused by the virus.

In various aspects, the present disclosure provides a pharmaceutical composition comprising a bacteriophage, wherein the bacteriophage is capable of binding to a virus with a dissociation constant of no more than 100 nM, and wherein the pharmaceutical composition is formulated for inhalation.

In some aspects, the pharmaceutical composition comprises at least 2, at least 10, at least 20, at least 50, or at least 100 bacteriophage variants capable of binding to the virus. In some aspects, the pharmaceutical composition comprises at least 50 bacteriophage variants capable of binding to the virus. In some aspects, the pharmaceutical composition is formulated for inhalation via nebulization.

In some aspects, the pharmaceutical composition comprises a concentration of from 10⁵ to about 10¹² bacteriophage particles per mL. In some aspects, the pharmaceutical composition comprises a concentration of from 10⁶ to about 10¹¹ bacteriophage particles per mL. In some aspects, the pharmaceutical composition comprises a concentration of from 10⁹ to about 10¹⁰ bacteriophage particles per mL.

In some aspects, the pharmaceutical composition is free of pathogens. In some aspects, the pharmaceutical composition sterile filtered. In some aspects, the pharmaceutical composition is free of pyrogens.

In various aspects, the present disclosure provides a sanitizing composition comprising a bacteriophage, wherein the bacteriophage is capable of binding to a virus with a dissociation constant of no more than 100 nM, and wherein the pharmaceutical composition is formulated for application to a surface.

In some aspects, the sanitizing composition comprises at least 2, at least 10, at least 20, at least 50, or at least 100 bacteriophage variants capable of binding to the virus. In some aspects, the sanitizing composition comprises at least 50 bacteriophage variants capable of binding to the virus. In some aspects, the sanitizing composition comprises a concentration of from 10⁵ to about 10¹² bacteriophage particles per mL. In some aspects, the sanitizing composition comprises a concentration of from 10⁶ to about 10¹¹ bacteriophage particles per mL. In some aspects, the sanitizing composition comprises a concentration of from 10⁹ to about 10¹⁰ bacteriophage particles per mL.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 schematically illustrates inhibition of viral entry into a host cell by binding of bacteriophages to the virus surface, thereby reducing the infectivity of the virus.

FIG. 2 illustrates a process of identifying a bacteriophage for treating or preventing a viral infection, producing the bacteriophage, and providing the bacteriophage to a patient.

FIG. 3 illustrates a method of identifying bacteriophages to treat or prevent a viral infection.

FIG. 4 schematically illustrates a method of screening for bacteriophages that bind to a virus.

FIG. 5 illustrates a method of producing bacteriophage for treating or preventing a viral infection.

### DETAILED DESCRIPTION

The present disclosure provides methods and compositions to for treating or preventing a viral infection in a eukaryote using bacteriophages that bind to the virus and prevent host cell invasion. A bacteriophage, also referred to herein as a “phage” or a “prokaryotic virus,” may interact with the surface of a virus, such as a virus capable of infecting a eukaryotic cell, referred to herein as a “eukaryotic virus.” In some embodiments, a viral infection may be treated or prevented by reducing the infectivity of the virus that causes the viral infection. The bacteriophages of the present disclosure may reduce the infectivity of the virus by binding to the virus and blocking interactions between the virus and a host cell. Also described herein are methods of identifying and producing compositions comprising bacteriophages that bind to viral surfaces and prevent the virus from invading a host cell. The methods of the present disclosure may include methods of producing large-scale quantities of bacteriophages that bind to a virus.

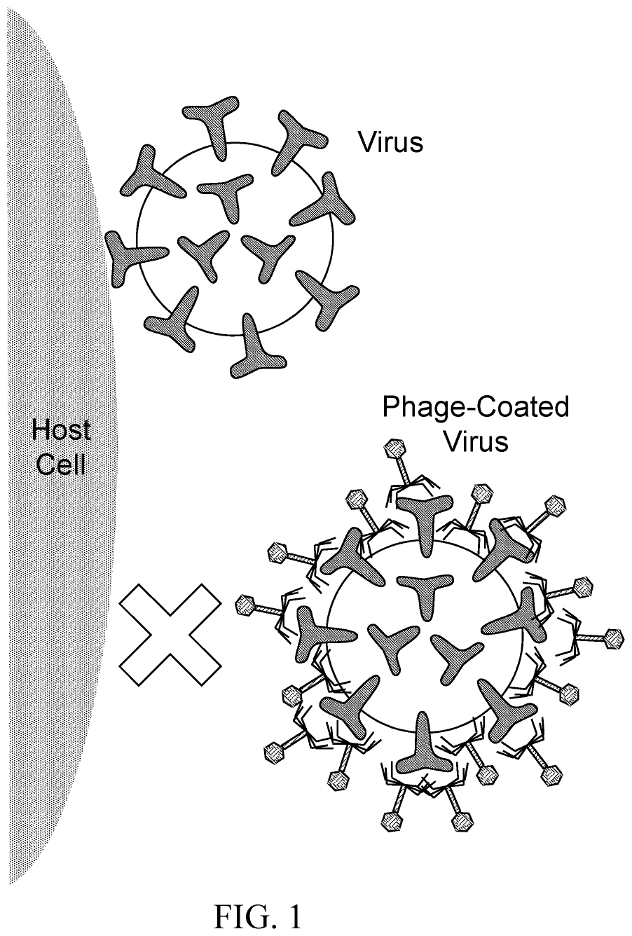

A virus may infect a host cell, such as a eukaryotic host cell, by interacting with the host cell surface through contacts between capsid or envelope proteins on the surface of the virus and receptor proteins on the host cell surface. Bacteriophages that bind to the virus may prevent viral infection of the host cell by blocking the interaction between viral surface proteins and host cell receptors, thereby reducing the infectivity of the virus, as illustrated in FIG. 1. In contrast to conventional bacteriophage therapy, in which bacteriophage infect and kill a bacterial host, the bacteriophages of the present disclosure coat the surface of a virus and prevent viral invasion of a host cell. A bacteriophage composition used for treating or preventing a viral infection, or reducing the infectivity of a virus, may offer additional advantages over conventional bacteriophage therapy. For example, bacteriophages that reduce the infectivity of a virus do not need to penetrate the surface of a pathogen or deliver genetic material into the pathogen in order to be effective. As a result, the bacteriophage compositions described herein may be less sensitive to pathogen mutation than conventional bacteriophage therapies that must infect bacterial cells to function.

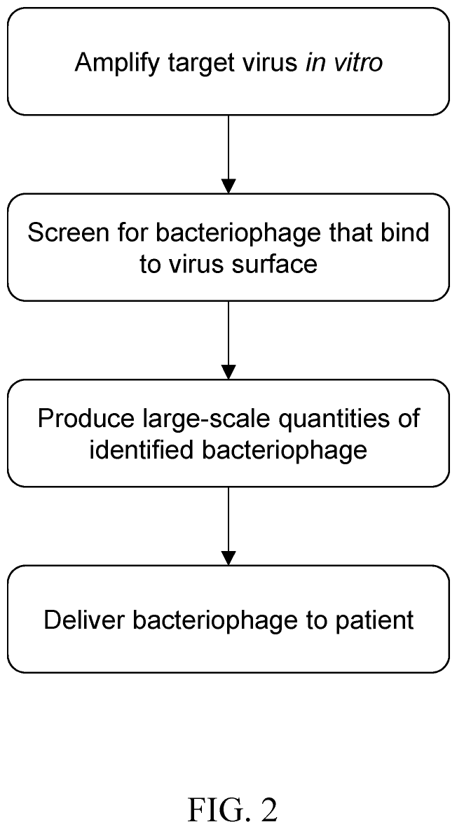

An example of a workflow for identifying a bacteriophage that inhibits viral invasion of a host cell and treating a patient with the identified bacteriophage is shown in FIG. 2. A sample of a virus of interest, such as a eukaryotic virus capable of causing disease in a eukaryotic organism, may be obtained from a virus sample repository. Examples of virus sample repositories include the Biodefense and Emerging Infections Resources Repository, the National Collection of Pathogenic Viruses, and the European Virus Archives. The virus may be amplified in vitro, for example in a eukaryotic cell culture, and isolated for use in bacteriophages screens. Bacteriophage may be screened for their ability to bind to the surface of the virus, for example using phage display screening methods. Identified bacteriophages with viral-binding properties may be produced in large-large scale quantities, for example in E. coli. The bacteriophages may be formulated for delivery to patients with the virus or at risk of exposure to the virus.

### Bacteriophage Screening, Production, and Compositions

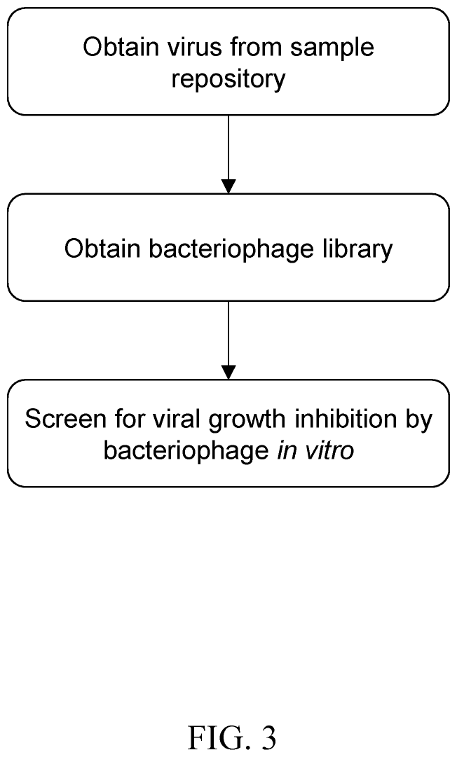

In some embodiments, the present disclosure provides methods of identifying a bacteriophage that binds to a virus, preventing the virus from invading a host cell. A method of identifying a bacteriophage that binds to a virus may comprise screening a phage library to identify high diversity bacteriophages that bind to the surface of a virus with high affinity. An example of a workflow for identifying a bacteriophage capable of treating or preventing a viral infection is shown in FIG. 3. A virus of interest may be selected and obtained from a sample repository, such as the Biodefense and Emerging Infections Resources Repository, the National Collection of Pathogenic Viruses, or the European Virus Archives. A bacteriophage library may be generated or obtained from a commercial source. In some embodiments, the bacteriophage library may be a λ phage, an M13 phage, a T4 phage, or a T7 phage library. The bacteriophage library may be screened for ability to bind to the virus of interest. As used herein, screening may also be referred to as “biopanning.” The identified bacteriophages may be further screened in vitro for their ability to disrupt interactions between the virus and a host cell, prevent viral invasion into the host cell, or to inhibit viral growth. The result of this screening process may be a selection of high-diversity bacteriophages that bind to a virus of interest with high affinity. A high-diversity selection of bacteriophages, containing multiple bacteriophages displaying different surface peptides that bind to the virus of interest, may be less sensitive to viral mutation than bacteriophages displaying the same surface peptide.

An identified selection of bacteriophages may be screened for the ability to disrupt interactions between the virus and a host cell, prevent viral invasion into the host cell, or to inhibit viral growth using a cultured cell line model. For example, the target virus may be amplified in a cell line (e.g., a mammalian cell line) and the bacteriophage collection may be applied to the cells and the amplified virus. The cultured cells and the amplified virus may be incubated with the bacteriophages for an amount of time sufficient for interactions between the bacteriophages and the virus to form. Interactions between the bacteriophage and the virus along with effects of the bacteriophage on viral invasion and growth may be observed by electron microscopy. In some embodiments, the bacteriophages may reduce the cytopathic effect of the virus on the cultured cells. An effective bacteriophage composition may reduce the cytopathic effect of the virus on the cells by at least about 10%, at least about 20%, at least about 30%, at least about 40%, at least about 50%, at least about 60%, at least about 70%, at least about 80%, at least about 90%, or about 100%. The selection of bacteriophages identified from the screening process may interact with the virus with higher frequency than a control bacteriophage that did not undergo the screening process.

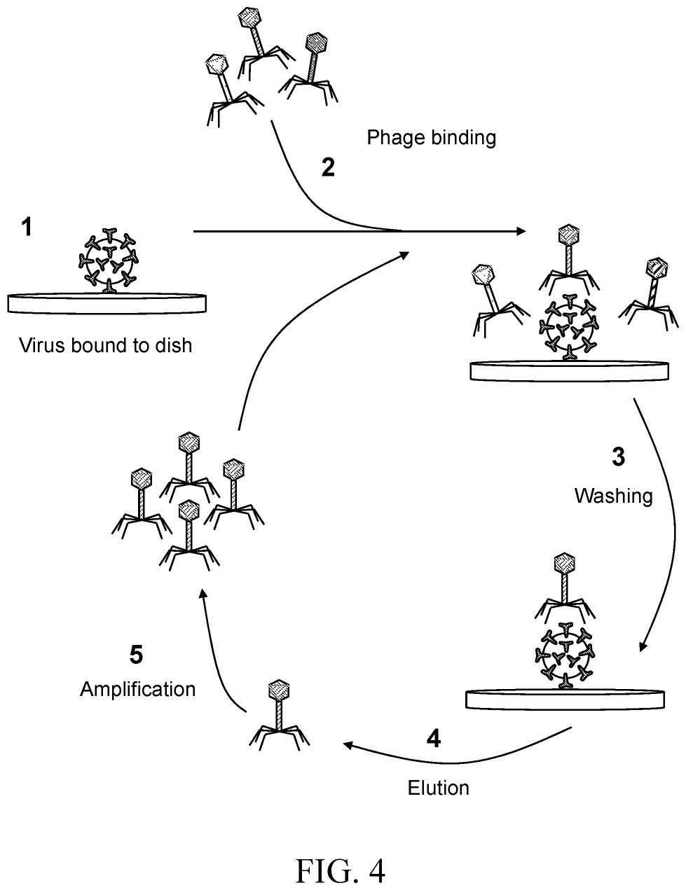

FIG. 4 illustrates an example of a method of screening for bacteriophages that bind to a target virus. At Step 1, a target virus is tightly attached to a surface, such as a dish, a plate, or a well. For example, the virus is covalently linked to the surface. In another example, the virus is adhered to the surface by a biotin-streptavidin interaction. A target virus may be any virus capable of causing disease in animal, including humans, or a plant. For example, a target virus may be any virus capable of causing disease in animal or plant. For example, a target virus may be a coronavirus (e.g., SARS-CoV-2, SARS-CoV, MERS-CoV, OC43, or HKU1), a human immunodeficiency virus, a herpes simplex virus, a hepatitis virus (e.g., hepatitis A, hepatitis B, or hepatitis C), a Marburg virus, an Ebola virus, a rhinovirus, an influenza virus, an avian influenza virus, a rotavirus, a norovirus, a dengue virus, a rabies virus, a mononucleosis virus, a human papillomavirus, a rubeola virus, a rubella virus, a zika virus, a varicella virus, or a poliovirus. In some embodiments, a target virus may be a virus causing diarrhea in humans, pigs, dogs, or cows, a virus causing fever and vasculitis in cats, a virus causing fever and anorexia in horses, a virus causing severe lung injury in mice, a virus causing lung disease and death from liver failure in whales and a virus causing respiratory tract infection in birds, such as bulbuls, sparrows, or chickens.

At Step 2, a phage library is contacted to the virus and allowed to bind to the virus. In some embodiments, a phage library may be obtained from a repository or a commercial source. Examples of a phage library include a filamentous phage library, a λ phage library, a T4 phage library, a T7 phage library, or an M13 phage library. In some embodiments, the phage library is a phage display library containing bacteriophages displaying from about 10⁷ to over 10¹² distinct peptides. The surface containing the virus is washed at Step 3 to remove any bacteriophage not tightly bound to the virus. The remaining tightly-bound viruses are eluted from the virus at Step 4, resulting in a collection of high-diversity, high-affinity bacteriophages. The resulting high-diversity, high-affinity bacteriophages bind to the virus with an average dissociation constant (KD) of no more than about 100 nM, no more than about 10 nM, or no more than about 1 nM. In some embodiments, the phages are eluted using a solution comprising a detergent or a high salt concentration. The eluted phages are amplified at Step 5. In some embodiments, the bacteriophages may be amplified in a bacterial host, such as E. coli. The amplified phages may be further screened to improve viral binding by repeating Steps 2 through 5.

Phage display screening methods that may be used to screen and identify bacteriophages are described in further detail in Ledsgaard et al., Toxins 2018, 10, 236; Harada et al., Microbiological Research 212-213 (2018) 38-58; and Deng et al., Molecular Medicine Reports 17: 714-720, 2018, which are herein incorporated by reference in their entirety. In contrast to these phage-display screening methods, in which peptides displayed on the phage surface are screened to identify proteins, such as antibodies, that may be used as therapeutic agents, the bacteriophages identified using the methods of the present disclosure are the therapeutic agent. The bacteriophage methods and compositions described herein may provide advantages over conventional antibody therapies for treating or preventing a viral infection, or reducing the infectivity of a virus. For example, a bacteriophage composition of the present disclosure may have improved stability for long-term storage compared to an antibody composition. In another example, a bacteriophage composition may be more cost-effective to produce in large quantities than an antibody composition.

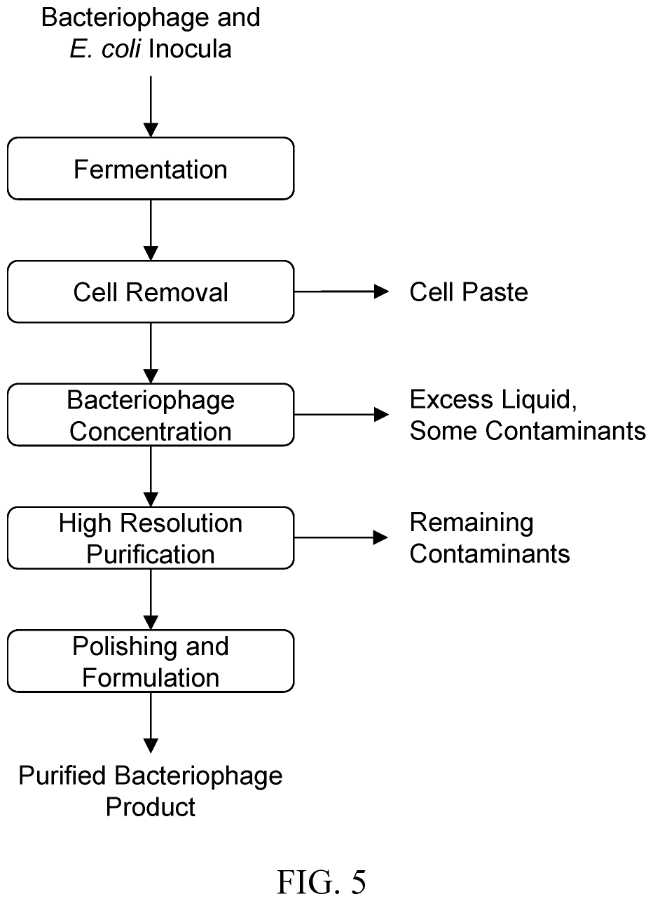

Bacteriophages identified by the screening methods described herein may be amplified and purified. An example of a workflow for large-scale amplification and purification of bacteriophages is illustrated in FIG. 5. A bacterial culture, such as E. coli, may be inoculated with a bacteriophage identified and purified using the methods described herein. In some embodiments, the bacterial culture may be inoculated with a high-diversity collection of bacteriophages encoded to display a variety of surface peptides. The inoculated bacterial culture may be fermented to grow the bacteria and amplify the bacteriophage. The bacterial cells may be removed as a cell paste, leaving the bacteriophage. The bacteriophage may be concentrated by removing excess liquid and some contaminants. The concentrated bacteriophage may be purified by high resolution purification, removing remaining contaminants. For example, high resolution may remove pathogens from the bacteriophage. In some embodiments, the column chromatography may be used to remove pyrogens from the bacteriophage composition. The purified bacteriophage may be polished and formulated for administration to a patient, resulting in a purified bacteriophage product. In some embodiments, the resulting bacteriophage composition may be free of pathogens. This method of amplification and purification may be used to obtain large quantities of bacteriophages, for example for medical use.

In some embodiments, a bacteriophage of the present disclosure may be modified with an additional antiviral agent. The additional antiviral agent may be connected to the bacteriophage via a linker. In some embodiments, the additional antiviral agent may be a protein covalent inhibitor adducts (PCIA) that covalently modifies viral proteins upon interaction between the virus and the modified bacteriophage. Examples of protein covalent inhibitor adducts that may be linked to the bacteriophage include N-ethylmaleimide, iodoacetamide, and N-phenylacrylamide. In some embodiments, the additional antiviral agent may be a chemically reactive group. The chemically reactive group may form a covalent bond with the virus (e.g., a viral protein), thereby stabilizing the interaction between the bacteriophage and the virus. In some embodiments, a peptide sequence displayed by the bacteriophage may serve as a linker to link the bacteriophage to the additional antiviral agent.

A bacteriophage composition of the present disclosure may comprise a bacteriophage displaying a peptide that interacts with a virus of interest. In some embodiments, a bacteriophage composition comprises bacteriophages displaying the same surface peptide. In some embodiments, a bacteriophage composition comprises high-diversity collection of bacteriophages displaying a variety of surface peptides that interact with a virus of interest. A high-diversity collection of bacteriophages may comprise multiple bacteriophage variants that bind to various regions, positions, or moieties on a viral surface. The peptides displayed by the high-diversity bacteriophages may interact with different viral surface proteins, proteoglycans, or lipids. In some embodiments, a high-diversity composition of bacteriophages may retain binding to a virus upon mutation of the virus. In some embodiments, the bacteriophage composition may comprise one or more covalently modified bacteriophages.

In some embodiments, a high-diversity collection of bacteriophages may comprise at least about 2, at least about 3, at least about 4, at least about 5, at least about 6, at least about 7, at least about 8, at least about 9, at least about 10, at least about 15, at least about 20, at least about 25, at least about 50, at least about 100, at least about 200, at least about 500, at least about 1000, or at least about 10,000 different bacteriophage variants capable of forming interacting with the virus of interest.

In some embodiments, a bacteriophage composition of the present disclosure is free of eukaryotic pathogens (e.g., bacteria or eukaryotic viruses). Pathogens may be removed from a bacteriophage composition by sterile filtration. Sterile filtration may be performed by passing an aqueous or liquid bacteriophage composition through a filter with a pore size large enough to allow passage of the bacteriophages but small enough to capture larger pathogens, such as bacteria and eukaryotic viruses. For example, a sterile filter to remove pathogens from a bacteriophage composition of the present disclosure may have a pore size of from about 30 nm to about 100 nm, from about 40 nm to about 100 nm, from about 50 nm to about 100 nm, from about 60 nm to about 100 nm, from about 70 nm to about 100 nm, from about 80 nm to about 100 nm, from about 90 nm to about 100 nm, from about 30 nm to about 120 nm, from about 40 nm to about 120 nm, from about 50 nm to about 120 nm, from about 60 nm to about 120 nm, from about 70 nm to about 120 nm, from about 80 nm to about 120 nm, from about 90 nm to about 120 nm, from about 30 nm to about 150 nm, from about 40 nm to about 150 nm, from about 50 nm to about 150 nm, from about 60 nm to about 150 nm, from about 70 nm to about 150 nm, from about 80 nm to about 150 nm, or from about 90 nm to about 150 nm.

The bacteriophages of the present disclosure may be formulated as a pharmaceutical composition for administration to a subject. The pharmaceutical composition may be administered to the subject to treat or prevent a viral infection. A pharmaceutical composition may be prepared by admixing a collection of bacteriophages with a pharmaceutically acceptable carrier. A pharmaceutical composition may further comprise one or more antioxidants, buffers, bacteriostats, solutes, suspending agents, solubilizers, thickening agents, stabilizers, or preservatives. In some embodiments, the pharmaceutical composition may be formulated for parenteral, intravenous, intranasal, oral, or topical administration, or for administration via inhalation.

A composition for administration via inhalation may be formulated as an aerosol for nebulized delivery. The aerosol may be stored under pressure with an acceptable propellant, such as dichlorodifluoromethane, propane, or nitrogen. A composition for oral administration may be formulated as a liquid solution, a capsule, a sachet, or a tablet, each containing a predetermined amount of the active ingredient, as liquids, solids, granules or gelatin; a liquid suspension, or an emulsion. A liquid formulation for oral delivery may comprise bacteriophage suspended in a diluent, such as water, saline, or polyethylene glycol. A tablet formulation for oral delivery may comprise one or more of lactose, sucrose, mannitol, sorbitol, calcium phosphates, corn starch, potato starch, microcrystalline cellulose, gelatin, colloidal silicon dioxide, talc, magnesium stearate, stearic acid, colorant, filler, binder, diluent, buffering agent, moistening agent, preservative, flavoring agent, dye, or disintegrating agent. A lozenge or pastille formulation for oral delivery may comprise one or more of a flavoring agent, such as sucrose, an inert base, such as gelatin or glycerin, or an emulsifier, such as acacia emulsions. A formulation for injection, such as by intra-articular, intravenous, intramuscular, intradermal, intraperitoneal, or subcutaneous routes, may comprise may be formulated as an isotonic sterile injection solution.

In some embodiments, a composition of the present disclosure may be formulated at a concentration of least about 10³, at least about 10⁴, at least about 10⁵, at least about 10⁶, at least about 10⁷, at least about 10⁸, at least about 10⁹, at least about 10¹⁰, at least about 10¹¹, or at least about 10¹² bacteriophage particles per 1 mL. In some embodiments, a composition of the present disclosure may be formulated at a concentration of least about 10³, at least about 10⁴, at least about 10⁵, at least about 10⁶, at least about 10⁷, at least about 10⁸, at least about 10⁹, at least about 10¹⁰, at least about 10¹¹, or at least about 10¹² plaque forming units (PFU) per 1 mL. In some embodiments, a composition of the present disclosure may be formulated at a concentration of from about 10³ to about 10¹², from about 10⁴ to about 10¹¹, from about 10⁵ to about 10¹¹, from about 10⁶ to about 10¹¹, from about 10⁶ to about 10¹¹, from about 10⁷ to about 10¹¹, from about 10⁸ to about 10¹¹, or from about 10⁹ to about 10¹¹ bacteriophage particles per 1 mL. In some embodiments, a composition of the present disclosure may be formulated at a concentration of from about 10³ to about 10¹², from about 10⁴ to about 10¹¹, from about 10⁵ to about 10¹¹, from about 10⁶ to about 10¹¹, from about 10⁵ to about 10¹²¹, from about 10⁷ to about 10¹¹, from about 10⁸ to about 10¹¹, or from about 10⁹ to about 10¹⁰ plaque forming units (PFU) per 1 mL.

An example of a formulation for administration via nebulization comprises about 3×10⁹ PFU/mL in a saline-magnesium buffer. A 5 mL dose of the nebulized formulation, comprising about 1.5×10¹⁰ PFU, may be administered to a subject to treat or prevent a viral infection.

In some embodiments, a bacteriophage composition may be formulated for in vitro application. For example, a composition of the present disclosure may be formulated for application onto a surface, such as a frequently touched surface, or onto an article of clothing. In some embodiments, a composition for in vitro application may be formulated as a spray, a wipe, a gel, a lotion, a cream, a rinse agent, or a soap.

A bacteriophage composition of the present disclosure may be formulated for stable storage for extended periods of time or under adverse conditions. In some embodiments, a bacteriophage composition may be lyophilized in the presence of carriers or bulking agents to improve storage life. In some embodiments, a bacteriophage composition may be stored under refrigeration (e.g., at about 4° C.) in trypticase soy agar or brain heart infusion broth or stored deeply frozen (e.g., at about −80° C.) with a cryoprotectant (e.g., glycerol, sucrose, or trehalose). In some embodiments, a bacteriophage composition may be freeze dried for long-term storage. Additional methods of formulation for storage or administration are described in further detail in Malik et al., Advances in Colloidal and Interface Science 2017, 249, 100-133, which is herein incorporated by reference in its entirety.

### Methods of Treating or Preventing a Viral Infection

In some embodiments, the present disclosure provides methods of treating or preventing a viral infection by administering to a subject a composition comprising bacteriophages that bind to the surface of the virus, thereby preventing or treating the viral infection. The bacteriophage composition may be administered in an amount sufficient to inhibit entry of the virus into a host cell of the subject. In some embodiments, the bacteriophage composition may be administered by inhalation (e.g., via nebulization), or by parenteral, intravenous, intranasal, oral, or topical administration. In some embodiments, the methods of the present disclosure may be independent of a host immune response in the subject. Instead, the bacteriophages of the present disclosure may prevent a viral infection by blocking interactions between a virus and a host cell of the subject, thereby preventing viral invasion of the host cell. In some embodiments, the bacteriophages may block viral invasion and stimulate a host immune response.

In some embodiments, treating a viral infection may comprise reducing the infectivity of a virus. A bacteriophage composition may reduce the infectivity of the virus by binding to the surface of the virus and inhibiting interactions between the virus and a host cell. The bacteriophage may slow the spread of the virus or alleviate symptoms associated with the virus. In some embodiments, administering a bacteriophage composition to the subject may shorten the duration of the viral infection by about 10%, about 20%, about 30%, about 40%, about 50%, about 60%, about 70%, or about 80%.

In some embodiments, preventing a viral infection may comprise reducing the infectivity of a virus. A bacteriophage composition may reduce the infectivity of the virus by binding to the surface of the virus and inhibiting interactions between the virus and a host cell. The bacteriophage may reduce the chance that a subject develops a viral infection following exposure to the virus. In some embodiments, administering a bacteriophage composition to the subject may reduce the chance of developing a viral infection by at least about 10%, at least about 20%, at least about 30%, at least about 40%, at least about 50%, at least about 60%, at least about 70%, at least about 80%, at least about 90%, or about 100% upon exposure to the virus.

In some embodiments, the subject may be a human, a non-human animal (e.g., a dog, a cat, a mouse, a rat, a cow, a horse, a sheep, a pig, a monkey, an ape, or an arthropod), a plant, or a single-celled eukaryote. In some embodiments, the virus may be a coronavirus (e.g., SARS-CoV-2, SARS-CoV, MERS-CoV, OC43, or HKU1), a human immunodeficiency virus, a herpes simplex virus, a hepatitis virus (e.g., hepatitis A, hepatitis B, or hepatitis C), a Marburg virus, an Ebola virus, a rhinovirus, an influenza virus, an avian influenza virus, a rotavirus, a norovirus, a dengue virus, a rabies virus, a mononucleosis virus, a human papillomavirus, a rubeola virus, a rubella virus, a zika virus, a varicella virus, or a poliovirus.

A bacteriophage composition may be administered to a subject in an amount sufficient to treat or prevent a viral infection. In some embodiments, a dosage may be determined by measuring a viral load in the subject and administering an amount of the bacteriophage composition based on the viral load of the subject. For example, a bacteriophage composition may be administered in an amount such that the number of bacteriophages administered is at least about 1-fold, at least about 2-fold, at least about 3-fold, at least about 5-fold, at least about 10-fold, at least about 12-fold, at least about 15-fold, at least about 20-fold, at least about 50-fold, at least about 100-fold, at least about 1000-fold, at least about 10,000-fold, or at least about 100,000-fold the number of virus particles present in the subject. In some embodiments, a dose of a bacteriophage composition sufficient to treat or prevent a viral infection may comprise at least about 10³, at least about 10⁴, at least about 10⁵, at least about 10⁶, at least about 10⁷, at least about 10⁸, at least about 10⁹, at least about 10¹⁰, at least about 10¹¹, or at least about 10¹² bacteriophage particles. For example, a dose of bacteriophage administered by inhalation as a nebulized formula may comprise from about 10⁷ to about 10¹⁰ bacteriophages.

In some embodiments, a bacteriophage composition may be administered orally along with or following administration of an antacid, such as calcium carbonate or bicarbonate, to neutralize stomach acid. In some embodiments, an oral composition may be encapsulated by an enteric coating, such as hydroxypropyl methylcellulose.

In some embodiments, the method of administration may be selected based on the target virus. For example, a bacteriophage composition to treat or prevent a respiratory virus (e.g., SARS-CoV-2 or SARS-CoV) may be administered by inhalation via nebulization. In another example, a bacteriophage composition to treat or prevent a blood-borne viral infection (e.g., HIV, hepatitis B, hepatitis C) may be administered intravenously. In another example, a bacteriophage composition to treat or prevent an intestinal virus (e.g., a virus causing viral gastroenteritis) may be administered orally.

In some embodiments, a method of preventing a viral infection may comprise applying a bacteriophage composition of the present disclosure in vitro. The bacteriophage composition may bind to a virus at the site of application and block entry of the virus into a host cell, thereby preventing a viral infection. A bacteriophage composition may be used as a disinfectant by applying the composition to a surface or substrate that may be at risk of accumulating virus. For example, a bacteriophage composition of the present disclosure may be applied to a frequently touched surface such as a handle (e.g., a door handle, a faucet, or a toilet handle), a button (e.g., an elevator button or a doorbell), a switch (e.g., a light switch or an appliance switch), a seat (e.g., a chair, a bench, or a toilet seat), a counter, a floor, a wearable article (e.g., a mask or gloves), an article of clothing, a body part (e.g., hands), or an object (e.g., a pen or an appliance), to decrease the infectivity of a virus on the surface. In some embodiments, the bacteriophage composition may be included in a cleaning solution, a coating solution, a cosmetic composition, a wearable substrate (e.g., a mask, gloves, or an article of clothing).

In some embodiments, preventing a viral infection may comprise reducing the infectivity of a virus. A bacteriophage composition may reduce the infectivity of the virus by binding to the surface of the virus and inhibiting interactions between the virus and a host cell. The bacteriophage may reduce transmission rate of a virus. In some embodiments, applying a bacteriophage composition to a surface may reduce the transmission rate of the virus by at least about 10%, at least about 20%, at least about 30%, at least about 40%, at least about 50%, at least about 60%, at least about 70%, at least about 80%, at least about 90%, or about 100%.

In some embodiments, a bacteriophage composition of the present disclosure may be used in a method of treating or preventing a viral infection in a plant. The composition may be applied to the surface of the plant, or the composition may be absorbed by the plant. For example, the composition may be added to the soil or aqueous solution around the plant, or the composition may be sprayed or poured onto the plant. The composition may treat or prevent a viral infection in the plant by blocking or inhibiting viral entry into a cell of the plant. In some embodiments, a bacteriophage composition of the present disclosure may be used to treat or prevent an infection caused by a tobacco mosaic virus, a tomato spotted wilt virus, a tomato yellow leaf curl virus, a cucumber mosaic virus, a potato virus, a cauliflower mosaic virus, an African cassava mosaic virus, a plum pox virus, or a brome mosaic virus.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

As used herein, the singular form “a”, “an”, and “the” includes plural references unless indicated otherwise.

As used herein, “pharmaceutically acceptable carrier” includes any and all solvents, dispersion media, coatings, antibacterial and antifungal agents, isotonic and absorption delaying agents and the like.

As used herein, the terms “treatment,” “treating,” and the like, covers any treatment of a disease in an animal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

### EXAMPLES

The invention is further illustrated by the following non-limiting examples.

### Example 1

### Screening a Phage Library for Bacteriophages that Bind and Inhibit a Virus

This example describes screening a phage library to identify bacteriophages that bind to a virus and inhibit viral entry. A virus sample is obtained from a virus repository. An M13 phage display library is obtained from a commercial vendor. The phage library is screened to selected for bacteriophages that bind to the virus. The virus is attached to the surface of a sterile dish through a biotin-streptavidin linkage. The surface of the dish is blocked with bovine serum albumin (BSA) or casein. The phage display library is contacted to the dish and incubated for sufficient time to allow the bacteriophages to bind to the virus. After incubation, the dish is washed with a buffered solution to remove bacteriophages that are not bounds to the virus or that are weakly bound to the virus. The remaining, tightly bound bacteriophages are eluted with a high-salt buffer. The resulting tightly-bound bacteriophages have a high affinity for the virus, binding the virus with an average dissociation constant (K_{D}) of no more than about 100 nM. A high-diversity formulation contains at least 10 distinct bacteriophage types with an average dissociation constant (K_{D}) of no more than about 100 nM.

The eluted bacteriophages are amplified in E. coli by infecting the E. coli with the eluted bacteriophages and culturing the E. coli. The cultured E. coli are lysed to release the amplified bacteriophages and centrifuged to separate the bacteriophages from the E. coli cells. The separated bacteriophages are filtered and concentrated to remove contaminants and excess liquid.

The amplified bacteriophages undergo a second round of screening. The filtered and concentrated bacteriophages are added to a fresh plate of virus. The binding, washing, elution, amplification, and separation steps are repeated. The amplified bacteriophages are purified by high resolution purification to remove remaining contaminants and sterile filtered to remove non-bacteriophage pathogens, including bacteria and viruses.

The ability of the bacteriophages to inhibit viral entry into a host cell is tested in Vero cells. Vero cells are contacted to the virus pre-incubated with the bacteriophage solution at different concentrations and the cytopathic effect of the virus on the Vero cells is measured. As a negative control, Vero cells are contacted to virus that has not been pre-incubated with bacteriophages. The ability of the bacteriophages to inhibit viral entry is determined by comparing the cytopathic effect of virus pre-incubated with bacteriophages to the cytopathic effect of the virus that was not pre-incubated with the bacteriophages. Bacteriophages that result in at least a 50% reduction in cytopathic effect are effective at inhibiting viral entry.

### Example 2

### Large-Scale Production of a Bacteriophage Composition

This example describes large-scale production of a bacteriophage composition for treating or preventing a viral infection. Bacteriophages are screened for the ability to bind to a virus and inhibit viral entry into a host cell, as described in EXAMPLE 1. The identified bacteriophages are amplified in E. coli by inoculating E. coli with the screened bacteriophages. The bacteriophages are amplified by large-scale fermentation of the inoculated E. coli cells. The E. coli cells are lysed and the bacteriophages are separated from the E. coli cells by centrifugation. The cell paste is removed, and the remaining solution containing the bacteriophages is centrifuged again and higher speed to concentrate the bacteriophages and remove additional contaminants. Additional purification is performed by column chromatography to remove additional contaminants, including pyrogens. Non-bacteriophage pathogens, including bacteria and viruses, are removed by sterile filtration. The resulting sterile and pyrogen-free bacteriophage composition is formulated for pharmaceutical administration by inhalation or injection. The final formulation contains a bacteriophage concentration of 3×10⁹ PFU/mL in a saline-magnesium buffer.

### Example 3

### Prevention of a Coronavirus Infection by Administering a Bacteriophage Composition

This example describes prevention of a coronavirus infection by administering a bacteriophage composition. Bacteriophages are screened for the ability to bind to and inhibit a coronavirus, as described in EXAMPLE 1, and a pharmaceutical composition of the bacteriophages is prepared as described in EXAMPLE 2. The resulting composition is administered by inhalation as a nebulized formulation to a human subject that has been exposed to a coronavirus. The subject is administered a dose of 5 mL of a high-diversity bacteriophage composition at a concentration of about 3×10⁹ PFU/mL, for a total dose of about 1.5×10¹⁰ bacteriophage particles. The bacteriophage composition decreases the infectivity of the virus by binding to the surface of the virus and preventing the coronavirus from invading the cells of the subject, thereby preventing the coronavirus infection. Administration of the composition decreases the chance of developing a coronavirus infection following exposure by at least 50%. By reducing the infectivity of the coronavirus in the subject, the infection is prevented.

### Example 4

### Treatment of a Coronavirus Infection by Administering a Bacteriophage Composition

This example describes treatment of a coronavirus infection by administering a bacteriophage composition to a subject with a coronavirus infection. Bacteriophages are screened for the ability to bind to and inhibit a coronavirus, as described in EXAMPLE 1, and a pharmaceutical composition of the bacteriophages is prepared as described in EXAMPLE 2. The resulting composition is administered by inhalation as a nebulized formulation to a human subject that has a coronavirus infection. A virus titer is measured in the subject, and a dose is determined based on the estimated number of virions present in the subject. The subject is administered a dose of bacteriophage particles that is approximately 10-fold the estimated number of coronavirus particles present in the subject. The composition is administered at a concentration of about 3×10⁹ PFU/mL, in an amount sufficient to treat the coronavirus infection. The bacteriophage treats the coronavirus infection by binding to the surface of coronaviruses in the subject, thereby reducing the infectivity of the virus and preventing the viruses from invading new cells in the host. Inhalation of the bacteriophage composition decreases the severity of respiratory symptoms caused by the coronavirus and shortens the length of illness by up to 50%. By reducing the infectivity of the virus in the patient, the patient is treated for the coronavirus infection.

### Example 5

### Prevention of a Rabies Infection by Administering a Bacteriophage Composition

This example describes prevention of a rabies infection by administering a bacteriophage composition. Bacteriophages are screened for the ability to bind to and inhibit a rabies virus, as described in EXAMPLE 1, and a pharmaceutical composition of the bacteriophages is prepared as described in EXAMPLE 2. The resulting composition is administered by intravenous injection to a human subject that has been exposed to a rabies virus. The subject is intravenously administered a dose of a high-diversity bacteriophage composition. The bacteriophage composition decreases the infectivity of the virus by binding to the surface of the virus and preventing the rabies virus from invading the cells of the subject, thereby preventing the rabies virus infection. Administration of the composition decreases the chance of developing rabies following exposure by at least 50%. By reducing the infectivity of the rabies virus in the subject, the infection is prevented.

### Example 6

### Prevention of an HIV Infection by Administering a Bacteriophage Composition

This example describes prevention of an HIV infection by administering a bacteriophage composition. Bacteriophages are screened for the ability to bind to and inhibit HIV, as described in EXAMPLE 1, and a pharmaceutical composition of the bacteriophages is prepared as described in EXAMPLE 2. The resulting composition is administered by intravenous injection to a human subject that has been exposed to HIV. The subject is intravenously administered a dose of a high-diversity bacteriophage composition. The bacteriophage composition decreases the infectivity of the virus by binding to the surface of the virus and preventing the HIV from invading the cells of the subject, thereby preventing the HIV virus infection. Administration of the composition decreases the chance of developing AIDS following exposure by at least 50%. By reducing the infectivity of the HIV in the subject, the infection is prevented.

### Example 7

### Prevention of a Hepatitis C Infection by Administering a Bacteriophage Composition

This example describes prevention of a hepatitis C infection by administering a bacteriophage composition. Bacteriophages are screened for the ability to bind to and inhibit a hepatitis C virus, as described in EXAMPLE 1, and a pharmaceutical composition of the bacteriophages is prepared as described in EXAMPLE 2. The resulting composition is administered by intravenous injection to a human subject that has been exposed to a hepatitis C virus. The subject is intravenously administered a dose of a high-diversity bacteriophage composition. The bacteriophage composition decreases the infectivity of the virus by binding to the surface of the virus and preventing the hepatitis C virus from invading the cells of the subject, thereby preventing the hepatitis C infection. Administration of the composition decreases the chance of developing hepatitis C following exposure by at least 50%. By reducing the infectivity of the hepatitis C virus in the subject, the infection is prevented.

### Example 8

### Prevention of an Ebola Virus Infection by Administering a Bacteriophage Composition

This example describes prevention of an Ebola virus infection by administering a bacteriophage composition. Bacteriophages are screened for the ability to bind to and inhibit an Ebola virus, as described in EXAMPLE 1, and a pharmaceutical composition of the bacteriophages is prepared as described in EXAMPLE 2. The resulting composition is administered by intravenous injection to a human subject that has been exposed to an Ebola virus. The subject is intravenously administered a dose of a high-diversity bacteriophage composition. The bacteriophage composition decreases the infectivity of the virus by binding to the surface of the virus and preventing the Ebola virus from invading the cells of the subject, thereby preventing the Ebola virus infection. Administration of the composition decreases the chance of developing Ebola following exposure by at least 50%. By reducing the infectivity of the Ebola virus in the subject, the infection is prevented.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of reducing the infectivity of a virus in a subject, the method comprising:
administering a composition comprising a bacteriophage to the subject,
binding the bacteriophage to the virus,
inhibiting invasion of the virus into a cell of the subject, and
reducing the infectivity of the virus.

2. The method of claim 1, wherein the virus is selected from the group consisting of a coronavirus, a human immunodeficiency virus, a herpes simplex virus, a hepatitis virus, a Marburg virus, an Ebola virus, a rhinovirus, an influenza virus, an avian influenza virus, a rotavirus, a norovirus, a dengue virus, a rabies virus, a mononucleosis virus, a human papillomavirus, a rubeola virus, a rubella virus, a zika virus, a varicella virus, and a poliovirus.

3. The method of claim 2, wherein the coronavirus is SARS-CoV-2, SARS-CoV, MERS-CoV, OC43, or HKU1.

4. The method of any one of claims 1-3, wherein the composition is administered by inhalation, parenteral administration, intravenous administration, intranasal administration, oral administration, or topical administration.

5. The method of any one of claims 1-4, wherein the composition is administered by inhalation via nebulization.

6. The method of any one of claims 1-5, comprising delivering at least 10⁵, at least 10⁶, at least 10⁷, at least 10⁸, at least 10⁹, at least 10¹⁰, at least 10¹¹, or at least 10¹² bacteriophage particles to the subject.

7. The method of any one of claims 1-6, comprising delivering at least 10⁹ bacteriophage particles to the subject.

8. The method of any one of claims 1-7, wherein the subject is a human, a non-human animal, a plant, or a single-celled eukaryote.

9. The method of any one of claims 1-8, wherein the composition comprises at least 2, at least 10, at least 20, at least 50, or at least 100 bacteriophage variants capable of binding to the virus.

10. The method of any one of claims 1-9, wherein the composition comprises at least 50 bacteriophage variants capable of binding to the virus.

11. The method of any one of claims 1-10, wherein the bacteriophage binds to the virus with an average dissociation constant of no more than 100 nM.

12. The method of any one of claims 1-11, wherein reducing the infectivity of the virus comprises inhibiting interactions between the virus and the cell of the subject.

13. The method of any one of claims 1-12, wherein reducing the infectivity of the virus comprises binding the bacteriophage to the virus.

14. The method of any one of claims 1-13, wherein the bacteriophage binds to a coat protein of the virus.

15. The method of any one of claims 1-14, further comprising treating an infection caused by the virus.

16. The method of any one of claims 1-14, further comprising preventing an infection caused by the virus.

17. A method of reducing the infectivity of a virus on a surface, the method comprising:
applying a composition comprising a bacteriophage to the surface,
binding the bacteriophage to the virus,
inhibiting invasion of the virus into a host cell, and
reducing the infectivity of the virus.

18. The method of claim 17, wherein the host cell is a eukaryotic host cell.

19. The method of claim 17 or claim 18, wherein the virus is selected from the group consisting of a coronavirus, a human immunodeficiency virus, a herpes simplex virus, a hepatitis virus, a Marburg virus, an Ebola virus, a rhinovirus, an influenza virus, an avian influenza virus, a rotavirus, a norovirus, a dengue virus, a rabies virus, a mononucleosis virus, a human papillomavirus, a rubeola virus, a rubella virus, a zika virus, a varicella virus, and a poliovirus.

20. The method of claim 19, wherein the coronavirus is SARS-CoV-2, SARS-CoV, MERS-CoV, OC43, or HKU1.

21. The method of any one of claims 17-20, wherein the composition comprises at least 2, at least 10, at least 20, at least 50, or at least 100 bacteriophage variants capable of binding to the virus.

22. The method of any one of claims 17-21, wherein the composition comprises at least 50 bacteriophage variants capable of binding to the virus.

23. The method of any one of claims 17-22, wherein the surface is a handle, a button, a switch, a seat, a counter, a floor, a wearable item, a body part, or an object.

24. The method of any one of claims 17-23, wherein the composition is applied to the surface at a concentration of from 10⁵ to about 10¹² bacteriophage particles per mL.

25. The method of any one of claims 17-24, wherein the composition is applied to the surface at a concentration of from 10⁶ to about 10¹¹ bacteriophage particles per mL.

26. The method of any one of claims 17-25, wherein the composition is applied to the surface at a concentration of from 10⁹ to about 10¹⁰ bacteriophage particles per mL.

27. The method of any one of claims 17-26, wherein the bacteriophage binds to the virus with an average dissociation constant of no more than 100 nM.

28. The method of any one of claims 17-27, wherein reducing the infectivity of the virus comprises inhibiting interactions between the virus and the host cell.

29. The method of any one of claims 17-28, wherein reducing the infectivity of the virus comprises binding the bacteriophage to the virus.

30. The method of any one of claims 17-29, wherein the bacteriophage binds to a coat protein of the virus.

31. The method of any one of claims 17-30, further comprising preventing an infection caused by the virus.

32. A pharmaceutical composition comprising a bacteriophage, wherein the bacteriophage is capable of binding to a virus with a dissociation constant of no more than 100 nM, and wherein the pharmaceutical composition is formulated for inhalation.

33. The pharmaceutical composition of claim 32, comprising at least 2, at least 10, at least 20, at least 50, or at least 100 bacteriophage variants capable of binding to the virus.

34. The pharmaceutical composition of claim 32 or claim 33, comprising at least 50 bacteriophage variants capable of binding to the virus.

35. The pharmaceutical composition of any one of claims 32-34, wherein the pharmaceutical composition is formulated for inhalation via nebulization.

36. The pharmaceutical composition of any one of claims 32-35, comprising a concentration of from 10⁵ to about 10¹² bacteriophage particles per mL.

37. The pharmaceutical composition of any one of claims 32-36, comprising a concentration of from 10⁶ to about 10¹¹ bacteriophage particles per mL.

38. The pharmaceutical composition of any one of claims 32-37, comprising a concentration of from 10⁹ to about 10¹⁰ bacteriophage particles per mL.

39. The pharmaceutical composition of any one of claims 32-38, wherein the pharmaceutical composition is free of pathogens.

40. The pharmaceutical composition of any one of claims 32-39, wherein the pharmaceutical composition sterile filtered.

41. The pharmaceutical composition of any one of claims 32-40, wherein the pharmaceutical composition is free of pyrogens.

42. A sanitizing composition comprising a bacteriophage, wherein the bacteriophage is capable of binding to a virus with a dissociation constant of no more than 100 nM, and wherein the sanitizing composition is formulated for application to a surface.

43. The sanitizing composition of claim 42, comprising at least 2, at least 10, at least 20, at least 50, or at least 100 bacteriophage variants capable of binding to the virus.

44. The sanitizing composition of claim 42 or claim 43, comprising at least 50 bacteriophage variants capable of binding to the virus.

45. The sanitizing composition of any one of claims 42-44, comprising a concentration of from 10⁵ to about 10¹² bacteriophage particles per mL.

46. The sanitizing composition of any one of claims 42-45, comprising a concentration of from 10⁶ to about 10¹¹ bacteriophage particles per mL.

47. The sanitizing composition of any one of claims 42-46, comprising a concentration of from 10⁹ to about 10¹⁰ bacteriophage particles per mL.
